(19) United States
(12) Patent Application Publication
(10) Pub. No.: US 2019/0290837 A1
(43) Pub. Date: Sep. 26, 2019

(54) Device for Thorough Delivery of Intravenous Medicine

(71) Applicant: Aaron Warta, Lexington, KY (US)

(72) Inventor: Aaron Warta, Lexington, KY (US)

(21) Appl. No.: 16/359,175

(22) Filed: Mar. 20, 2019

Related U.S. Application Data
(60) Provisional application No. 62/645,954, filed on Mar. 21, 2018.

(51) Int. Cl. A61M 5/142; A61M 5/168; A61M 5/14; A61M 39/10

(52) U.S. Cl. CPC A61M 5/142; A61M 5/168; A61K 9/0019; A61M 39/1011; A61M 5/1414; A61M 5/1411

(57) ABSTRACT

A device for the thorough delivery of liquid medicine in an intravenous (IV) delivery system. The device consists of a negative pressure chamber filled with saline solution that is used to flush all of the residual liquid medicine from a secondary liquid medicine delivery tube. The negative pressure chamber sits below the drip chamber attached to the secondary medicine bag and is attached to the secondary medicine delivery tube in such a way that when the liquid medicine from the delivery tube has flowed below the negative pressure chamber, the negative pressure chamber releases the stored saline to flush the liquid medicine from the secondary liquid medicine delivery tube.

### CROSS REFERENCE TO RELATED APPLICATIONS

This application claims priority to U.S. provisional application Ser. No. 62/645,954, filed on Mar. 21, 2018, and incorporated herein by reference.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates to a negative pressure chamber designed to ensure complete delivery of intravenous medication in an intravenous IV medicine delivery system.

### Description of the Related Art

Saline intravenous drip bags are well known in the art and have been in use for many years. The saline drip bag delivers a saline solution (typically purified water with NaCl 0.9%) to the patient. The saline drip bag is held on a post or frame above the patient, with a primary tube that runs to the patient and that ends in a needle or cannula that is inserted into the patient. The needle or cannula is inserted directly into one of the patient's veins, typically at the inside of the elbow or the back of the hand. The process is known as intravenous delivery, since it delivers the solution directly into the patient's vein. The saline solution, or other liquids delivered through this system, is delivered into the patient by means of gravity.

There are two primary purposes for the saline drip bag. The first is to help provide liquids to the patient, and ensure the patient is properly hydrated. The second purpose of the saline drip bag and IV system is to ease and expedite delivery of medicine to the patient. The use of the IV system allows the patient to have a single needle, often called a catheter, inserted into the vein, and then to have a variety of medicines or other liquids delivered directly into the patient through the IV system. This means that medical staff only have to insert a needle into the patient's body a single time. The use of IV for delivery of medicine is also preferred since it allows the medicine to be inserted directly into the blood stream, and this allows it to be distributed throughout the body by normal circulation.

In many situations medicine is also delivered to the patient in conjunction with the primary solution bag. This is typically accomplished by the addition of a secondary drip bag that will be attached above the primary bag, with a secondary tube that runs from the secondary bag and attaches to the primary tube below the primary bag. The secondary tube is attached to the primary tube with a Y-site attachment port. Such Y-site attachment ports are well known in the art. This allows the medicine to flow from the secondary bag to the primary tube and into the patient. Because of gravity and hydraulic pressure, the solution from the secondary bag will flow into Y-site before the saline solution. The secondary solution with the medicine will flow into the primary tube until the secondary bag empties, and until the fluid in the secondary tube is parallel to the fluid in the primary bag. At that point hydraulic pressure will reverse the process, and the liquid in the primary bag will flow, leaving a portion of the medicine that remains in the secondary tube unused. The unused medicine remaining in the secondary tube is typically referred to as the residual volume. This is simply wasted medicine, and this waste of medicine can be both costly and inefficient. There is a need, therefore, for a way to eliminate this residual volume and ensure that all of the medicine from the secondary drip bag is fully used.

### SUMMARY OF THE INVENTION

This invention consists of a small cylindrical negative pressure chamber that is located below the secondary drip bag containing liquid medicine. The negative pressure chamber is designed to ensure that all of the liquid medicine flows out of the secondary liquid medicine bag and then to release the saline within the negative pressure chamber to flush all of the residual liquid medicine from the secondary delivery tube and thereby ensure that all of the liquid medicine is fully used.

### BRIEF DESCRIPTION OF THE DRAWINGS

FIG. 1 is a view of a typical prior art primary and secondary drip bag configuration.

FIG. 2 is a partially cut-away side view of the main components of the invention.

FIG. 3 is a view of the invention integrated with a standard primary and secondary drip bag configuration.

FIG. 4 is a perspective view of the negative pressure chamber and one embodiment of the air opening.

FIG. 5 is a side view of the negative pressure chamber with liquid therein and with the vertically intersecting tube terminating within the conical extension.

### DETAILED DESCRIPTION OF THE INVENTION

Detailed embodiments of the present invention are disclosed herein. It is to be understood that the disclosed embodiments are merely exemplary of the invention, and that there may be a variety of other alternate embodiments. The figures are not necessarily to scale, and some features may be exaggerated or minimized to show details of particular components.

Therefore, specified structural and functional details disclosed herein are not to be interpreted as limiting, but merely as a basis for teaching one skilled in the art to employ the varying embodiments of the present invention.

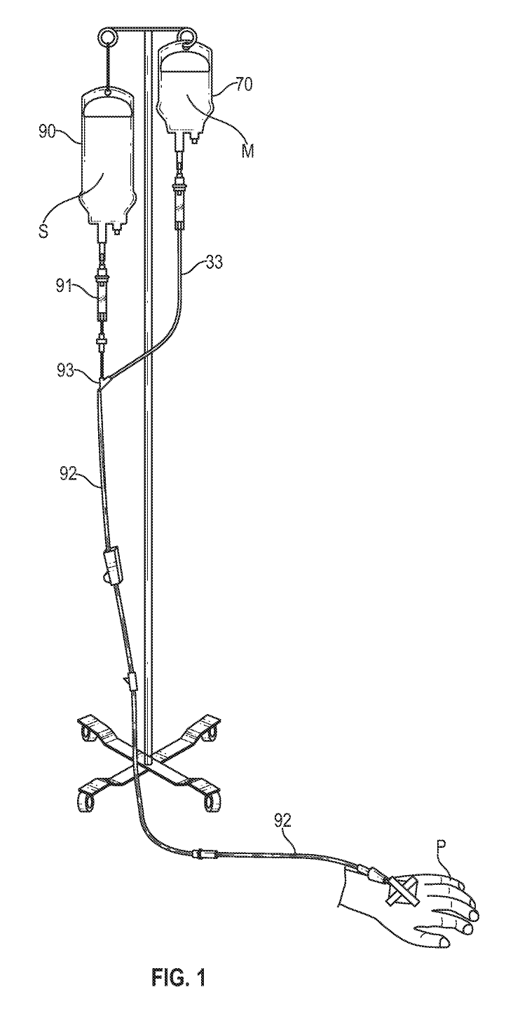

The present invention is designed for use in conjunction with a standard IV delivery system. FIG. 1 depicts a typical IV medicine delivery system. The main component of the system is a primary IV drip bag 90 which is filled with a saline solution S, which is typically purified water with 0.9% NaCl. The primary IV drip bag is typically made of a soft PVC or other plastic and is therefore subject to the forces of ambient air or atmospheric pressure. In a standard system there is a drip chamber 91 located below the primary bag. The drip chamber 91 allows the medical staff to visually monitor the flow rate of the saline solution, and is also designed to ensure that air does not enter the liquid delivery system, which can be dangerous to the patient. There is a primary IV tube 92 that begins at the bottom of the drip chamber 91, and terminates at the catheter in the patient P. Typically there are a number of slide clamps and roller clamps located on the primary tube 92 that allow medical personnel to control the delivery rate of fluids, including liquid medicine, to the patient. These slide clamps and roller clamps are standard and well known in the art. There is also typically one or more Y-shaped attachment ports that allow a second IV line to be attached to the primary tube 92. In the present invention the Y-shaped attachment port connector is known as the Y-site 93, which is located on the primary tube 92. The Y-site 93 allows the secondary delivery tube 33 to be attached to the primary tube 92 to allow the liquid medicine M from the secondary bag 70 to be introduced into the system and delivered to the patient P. The primary tube 92 runs to the patient P. In the standard IV delivery system the patient P has a needle or cannula inserted into a vein and an attachment port located externally so that doctors or medical staff can attach the IV tubing 92 to allow liquids to be introduced into the patient.

The standard IV system relies upon ambient air pressure, also referred to as atmospheric pressure, and gravity to ensure the flow and delivery of the liquids, both the saline solution S and the liquid medicine M. Both the primary bag 90 and the secondary bag 70 are made of soft plastic and so the liquids contained within the bags are subject the forces of atmospheric pressure. Because of the importance of the forces of gravity, the system described herein is oriented within standard spatial orientation, with up meaning away from the floor or ground, down meaning towards the floor or ground, above meaning higher than another element above the ground, and below meaning lower than another element toward the ground. The liquid in the system, meaning the saline solution S in the primary bag and the liquid medicine M in the secondary bag, flow downwardly due to gravity. The effects of gravity, and hydraulic pressure—which is a combination of the forces of atmospheric pressure and gravity on the liquids within the system—means that the liquids within the system will, whenever, possible, seek to achieve equal hydraulic pressure. The exception to this occurs in the negative pressure chamber 20, as described below. In the typical intravenous system the patient P receives a steady infusion of saline at a rate of between 5 ml and 20 ml per hour, which is referred to as the “keep vein open” rate. This means that the primary saline bag 90 is always connected and always flowing. Some systems also can be attached to an infusion pump that is located on the primary line 92, and that can more precisely control the rate of flow of saline or medicine. The use of an infusion pump has no impact on this invention.

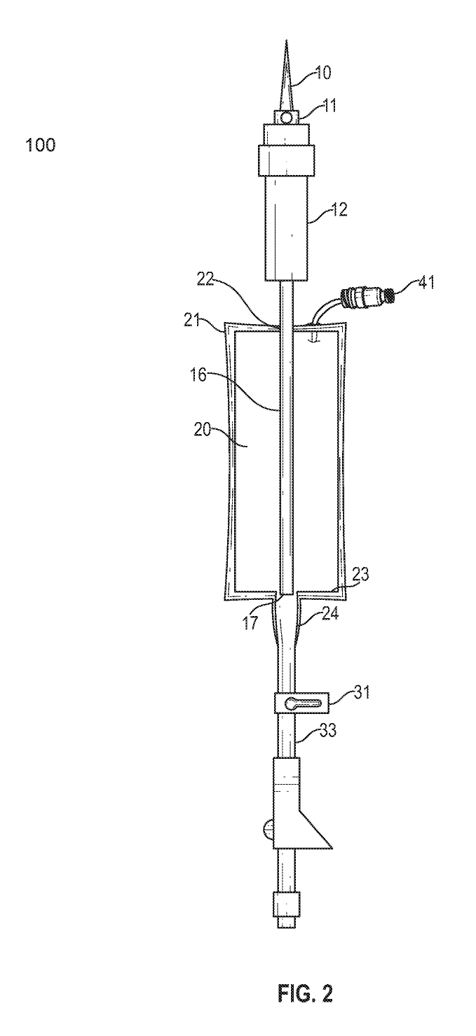

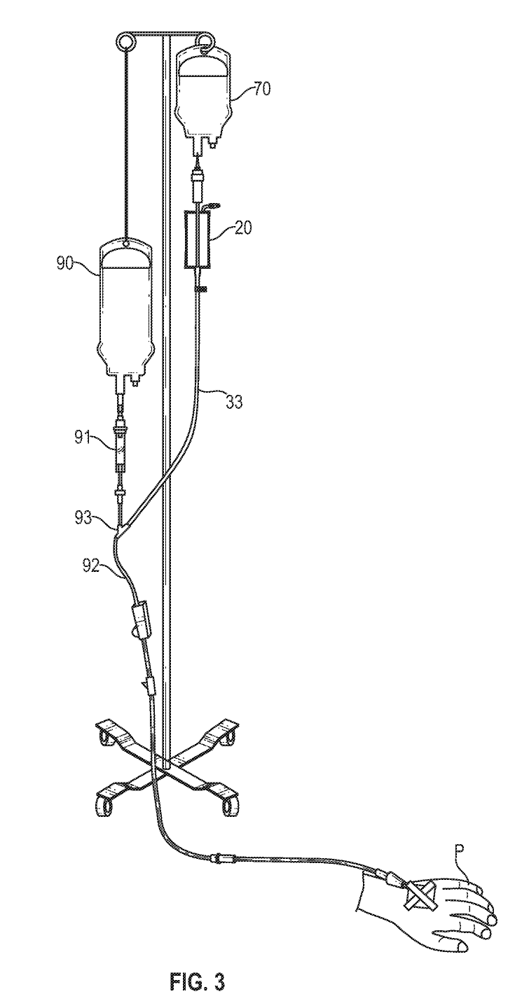

The thorough medicine delivery system 100 of the present invention is attached below the secondary IV bag 70, which contains liquid medicine M. FIG. 2 shows the components of the medicine delivery system 100 in detail, and FIG. 3 shows the medicine delivery system 100 integrated with the prior art liquid delivery system shown in FIG. 1. The components of FIG. 2 and FIG. 3 are not to scale but sized in the drawings to help explain the system. The secondary bag 70 is typically located 9 to 12 inches above the primary bag, which allows the forces of gravity to force the flow of the liquid medicine M, and the affinity of liquid to flow in the direction of least resistance, to allow the liquid medicine M to flow into the patient P before the primary saline solution S. The medicine delivery system 100 consists of a spike 10 that is inserted into a secondary solution IV drip bag 70. The spike 10 is a standard and well-known medical liquid delivery spike that includes internal passageways that allow the liquid medicine M in the secondary solution bag 70 to be introduced into the system 100. The secondary solution IV drip bag 70 is also a standard secondary solution bag, which is well known in the art. Secondary solution bags 70 typically have one or two access ports on the bottom of the bag to allow the attachment of delivery devices such as the standard spike 10. The access port often is closed with a thin membrane that is pierced by the spike, and the access port is sized to allow the introduction of the spike 10, and the internal passageway within the spike allows the liquid medicine M from the secondary bag 70 to be introduced into the system. The spike 10 includes an air vent 11, which is standard, and which allows air into the system to allow the liquid medicine M to flow into the drip chamber 12. The air vent 11 allows air to flow into the secondary bag 70 through the air passageway within the spike 10, which helps the liquid medicine M flow out of the secondary bag 70, through the spike 10, and into the drip chamber 12. The air vent 11 is also important when the liquid medicine M is in a glass bottle, since the glass bottle, unlike the secondary solution bag 70, is not pliable and therefore not subject to air pressure to help flow of the liquid medicine M. The drip chamber 12 is also a standard IV drip chamber that allows medical personnel to view the rate of the drip of the liquid medicine M and therefore the rate of the delivery of the liquid medicine M to the patient. There is a standard discharge nozzle at the bottom of the drip chamber 12 that allows the attachment of a standard IV tube to allow the liquid medicine M to flow into the IV system. The discharge nozzle at the bottom of the drip chamber 12 is sized and configured to attach to conventional IV tubing. In many cases the spike and drip chamber are a single component. Standard IV tubing, which is used in this system, is medical grade PVC or plastic tubing that is of a standardized size, typically having a 3 mm or a 4 mm internal diameter. All of the components of the present invention, except for the negative pressure chamber 20 and its components, are standard medical equipment and are designed to interconnect with all standard IV tubing and equipment. Typically, this equipment can be used with either 3 mm or 4 mm tubing, since the tubing is flexible and therefore can be attached to any standard IV equipment.

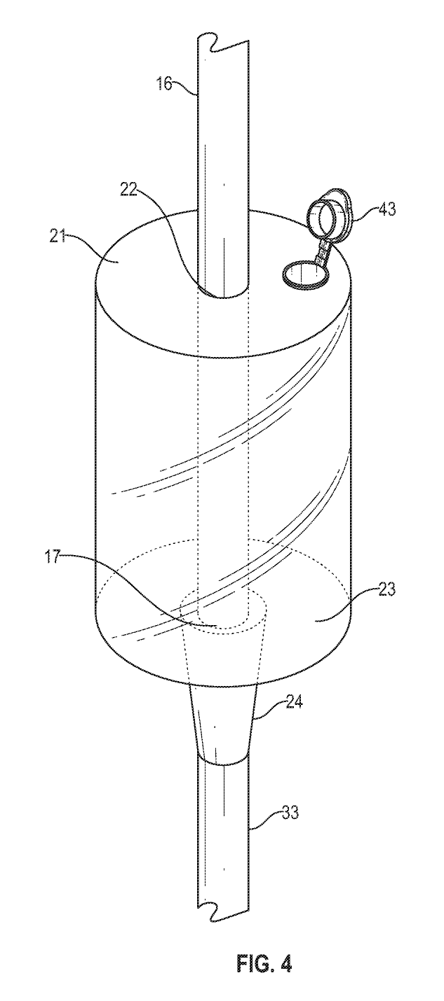

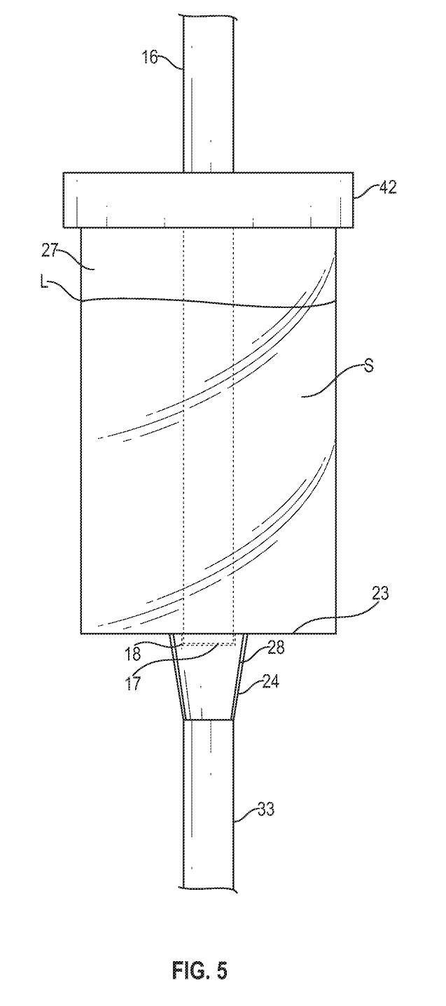

A standard IV tube is attached to the discharge attachment at the bottom of the drip chamber 12 and then runs into the negative pressure chamber 20. This tube will be referred to herein as the vertically intersecting tube 16. The vertically intersecting tube 16 runs internally through the negative pressure chamber 20. The negative pressure chamber 20 is shown in detail in the perspective view of FIG. 4, and shown with liquid L in the system in FIG. 5.

The negative pressure chamber 20 is an enclosed, hard, cylindrical compartment, which is intersected in the center by the vertically intersecting tube 16. The negative pressure chamber 20 is made of hard and non-pliable plastic to prevent the forces of atmospheric pressure from acting on the liquids within the negative pressure chamber 20. The negative pressure chamber 20, like most of the components in the IV system, is clear to allow medical personnel to observe the operation of the system and the flow of the liquids and liquid medicine M. In the preferred embodiment this is #5 plastic or polypropylene, but any clear, hard, medical grade plastic can be used. In the preferred embodiment the negative pressure chamber 20 is approximately the size of an 8 Dram pill vial, which equates to 29.57 milliliters in volume. The dimensions of the negative pressure chamber 20, in the preferred embodiment is 66 mm (2.598 inched) high, with a 28 mm (1.102 inch) diameter top. The size of the negative pressure chamber 20 can vary but in all configurations it is a short cylindrical chamber with a cylindrical side wall, a top 21 and a bottom 23. There is a chamber opening 22 located in the center of the top 21. The chamber opening 22 is a circular hole that is sized to allow the vertically intersecting tube 16 to enter the negative pressure chamber 20, and the chamber opening 22 is sized to snuggly and securely encircle the vertically intersecting tube 16 such that the seal between the vertically intersecting tube 16 and the chamber opening 22 is air tight. Standard IV tubing is made of soft, flexible, and pliable material (typically soft pliable polyvinyl chloride), and it is common to make openings that are sized to allow the IV tubing to be inserted, but to maintain an air tight seal. It is possible, and within the conception of the invention, to also include a standard grommet to create the air tight seal between the vertically intersecting tubing 16 and the chamber opening 22.

There are three embodiments of an air opening that allow the negative pressure chamber 20 to be opened to the air during the set-up of the system, but then resealed to be air-tight, and to separate the negative pressure chamber 20 from normal atmospheric pressure. As described herein, the three air openings are an air-tight lid 42, a needleless connection with a Luer lock 41, and a snap-open cap 43. In the preferred embodiment, shown in FIG. 5, the top 21 is an air-tight lid 42 that can be removeably attached to the top 21. The air-tight lid 42 is circular and is sized and configured to close the top 21. This can be in any conventional manner, but screwing and the common pill bottle twist cap are the most common. In these configurations the air-tight lid 42 can include an integrated seal inside the lid, which is typically made of silicon or other soft and flexible material such as rubber, so that as the air-tight lid 42 is screwed on, or otherwise attached, the seal in the air-tight lid 42 creates as air-tight seal between the air-tight lid 42 and the sides of the negative pressure chamber 20. When an air-tight lid 42 is used, the chamber opening 22 is located in the middle of the air-tight lid 42. These pill-bottle air tight lids are common and well know. In another embodiment, shown in FIG. 2, there is a standard Luer lock 41 with a twist open cap that is attached to, and through, the top 21 of the negative pressure chamber 20. Luer locks with twist open caps (as disclosed in U.S. Pat. No. 5,676,346) are well known in the art. In this configuration the Luer lock 41 can be open to the air by twisting the cap, typically clockwise to open, and then twisting counterclockwise to close. These types of twist caps are designed to provide an air-tight seal. In a third embodiment, shown in FIG. 4, the air opening is a standard small snap-open cap 43 attached to the top 21 of the negative pressure chamber 20. This type of snap-open cap 43 is made of conventional plastic and is well known in the art. The snap-open cap 43 can be easily flipped open to open the negative pressure chamber 20 to the ambient air, and then snapped closed to close off the negative pressure chamber 20 from ambient air. The purpose of the air opening is described below in association with the use of the device.

At the bottom 23 of the negative pressure chamber 20 there is a conical extension 24, which is conical shaped and extends downwardly from the suction chamber 20. The conical extension 24 is aligned with the center point of the bottom 23 of the negative pressure chamber 20. In the preferred embodiment the conical extension 24 has an approximately 10 mm inner diameter and is approximately 20 mm in length, and tapers from the 10 mm diameter down to a diameter to allow attachment of a standard IV tube, which is either 3 mm or 4 mm depending upon the tubing used throughout the system. The inner wall 28 of the conical extension 24 can taper down in straight line, leaving the inner wall 28 flat and conical, or can be curved downwardly, narrowing more at the end than at the top. Either configuration will accommodate the operation of the invention. The conical extension 24 can be made from the same material as the negative pressure chamber 20 and in one embodiment is molded as a single piece with the negative pressure chamber 20. In other embodiments it can be made of different material and attached to the bottom of the negative pressure chamber 20. The terminal end 17 of the vertically intersecting tube 16 terminates inside the conical extension 24 just below the bottom 23 surface of the suction chamber 20, as shown in FIG. 5. Since the intersecting tube 16 runs along the centerline of the tubular shaped negative pressure chamber 20, and since the conical extension 24 is positioned at the center of the bottom 23 of the negative pressure chamber 20, the intersecting tube 16, and more importantly the terminal end 17 of the intersecting tube 16 sit in the center of the conical extension 24, which means that there is a circumferential gap between the outer wall 18 of the terminal end 17 and the inner wall 28 of the conical extension 24. In the preferred embodiment the terminal end 17 is 5 mm below the bottom 23 and down into the conical extension 24. The positioning of the terminal end 17 inside the conical extension 24, just below the bottom 23 of the negative pressure chamber 20, is important to the operation of the negative pressure chamber 20. In the most preferred embodiment the terminal end 17 is 5 mm below the bottom 23. The outer diameter of the terminal end 17 will be 5 mm for a 3 mm tube and 6 mm for a 4 mm tube (the walls of the tube are 1 mm thick). This means that there is a clearance of roughly 2 to 2.5 mm between the outer wall 18 of the terminal end 17 and the inner wall 28 of the conical extension 24. This is best seen in FIG. 5. In the preferred embodiment, the gap between the inner wall 28 and the outer wall 18 is between 2 mm and 2.5 mm, but the system will work with other sized gaps, ranging from as small as 1 mm to as large as 6 mm.

The conical extension 24 is attached at the bottom to the secondary delivery tubing 33, which delivers the liquid medicine M or saline solution S to the patient P. The conical extension 24 tapers down to a sufficiently narrow size to allow the standard delivery tubing 33 to slide over the bottom end of the conical extension. Typically, after leaving the conical extension 24 the delivery tubing 33 is attached to the Y-site 93, so that the liquid medicine M flows from the delivery system 100 then to the primary tube 92 and on to the patient P.

In use, the negative pressure chamber 20 is partially filled with a saline solution S, which is the standard 0.9% NaCl with purified water solution. This can be seen in FIG. 5. The negative pressure chamber 20 can be filled in a number of ways. The most common is to attach the delivery tube 33 to the y-site 93, and then open the negative pressure chamber 20 to the air, by removing the lid 42, opening the Luer lock 41, or opening the snap-open cap 43. The next step is to hold the negative pressure chamber 20 below the liquid level in the primary drip bag 90. Because of the hydraulic pressure of the liquid in the primary bag 90, the liquid saline solution S will flow through the primary IV tube 92, through the y-site 93, into the secondary delivery tube 33, up the secondary delivery tube 33, and into the negative pressure chamber 20. The medical personnel who is operating the system can monitor the flow the liquid saline solution S into the negative pressure chamber 20, and when it reaches the desired liquid level L the technician can cease the flow of the liquid saline solution S by either raising the negative pressure chamber 20 so that the liquid saline S within the negative pressure chamber 20 is at the same level as the liquid in the primary bag 90, or by closing one of the air openings—the needleless connection Luer lock 41, the lid 42, or the snap-open cap 43. It is also possible, and within the conception of the invention, to draw the liquid saline solution S into the negative pressure chamber 20 by means of suction, typically with a large syringe. At this point the negative pressure chamber 20 can be sealed, by either attaching the lid 42, closing the needleless connection port 41, or the snap-open cap 43. Once the negative pressure chamber 20 is sealed the negative pressure chamber 20 will be separated from normal atmospheric pressure and this will prevent the saline solution S in the negative pressure chamber 20 from flowing outward, even if the liquid level L within the negative pressure chamber 20 is above the liquid level in the primary drip bag 90. This is the same physical phenomenon as holding a finger over the top end of a straw which will keep the liquid within the straw. The liquid stays in the straw because the finger over the top of the straw prevents normal atmospheric pressure from pushing the liquid down, and the capillary forces present between the liquid and the walls of the straw are stronger than the forces of gravity. The same physical phenomena are occurring here. With the negative pressure chamber 20 sealed, atmospheric pressure is not pressing down on the saline solution S, and the small clearance between the terminal end 17 of the intersecting tube 16 and the sidewalls of the conical extension 24, which is typically less than 3 mm, ensures that the capillary forces are sufficient to overcome the forces of gravity and to hold the saline solution S in the negative pressure chamber 20. It is important to note that the remainder of the system is still subject to ambient air pressure. The air vent in the drip chamber below the primary bag 90, as well as the air vent 11 in the drip chamber 12 below the secondary medicine bag 70 is open to ambient air, so the liquids in the system are fully subject to the effects of air pressure.

The second force that holds the liquid within the straw is known as the capillary force. The capillary force is a product of the intermolecular forces between the liquid and the walls of the components. It is well known that liquids like water can be drawn up against the forces of gravity within very small inner diameter tubes simply by these capillary forces. The Young-Laplace equation explains the capillary force and notes that it is, in part, a product of the surface tension between the liquid and the surfaces surrounding the liquid. In the aforementioned straw, the surface tension between the interior wall of the straw and the water in the straw is enough to overcome the forces of gravity and help retain the liquid within the straw. The same phenomena occurs within the conical extension 24 due to the surface area of the outer wall 18 of the terminal end 17 of the intersecting tube 16, and the inner wall 28 of the conical extension 24. The terminal end 17 descends 5 mm into the conical extension 24, and there is a 2 mm to 2.5 mm gap between the external wall of the terminal end 17 and the interior wall of the conical extension 24. When combined with the lack of ambient air pressure on the liquid saline S within the negative pressure chamber 20, this narrow gap between the two walls provides sufficient surface tension to hold the liquid saline S within the negative pressure chamber 20.

The term “negative pressure” as used in negative pressure chamber 20 is a slight misnomer because the pressure in the negative pressure chamber 20 is not negative. But it is less than the ambient atmospheric pressure acting on the rest of the system so it is negative in relation to ambient pressure, and so the term negative pressure is used. When exposed to atmospheric pressure all of the liquid in the standard IV delivery system will flow downward and will seek equilibrium, because all parts of the system are subject to the forces of gravity and atmospheric pressure. When the negative pressure chamber 20 is filled with liquid saline solution S and then sealed, it is no longer subject to atmospheric pressure and so the liquid will not flow downward.

Next the spike 10, drip chamber 12, and negative pressure chamber 20 with the saline solution S therein, is raised up to the secondary bag 70. The air vent 11 below the spike 10 will be open so that the liquid medicine M in the secondary bag 70, the spike 10, and the drip chamber 12 will be subject to normal atmospheric pressure. The negative pressure chamber 20 should be at a higher level than the primary bag 90 so that the liquid medicine M will from through the system before the saline solution S. This is shown in FIG. 3. Next the spike 10 is inserted into the secondary bag 70, and the liquid medicine M is allowed to flow into the drip chamber 12, and from there into the vertically intersecting tube 16. Since the secondary bag 70 is above the primary bag 90, gravity and hydraulic pressure will allow the liquid medicine M to flow into the vertically intersecting tube 16. When the liquid medicine M reaches the terminal end 17 of the vertically intersecting tube 16 it will flow into the conical extension 24. At this point gravity and hydraulic pressure will ensure that the liquid medicine M will flow down through the vertically intersecting tube 16, out of the terminal end 17, into the conical extension 24, and then into the delivery tubing 33, which takes the liquid medicine M down to the y-site 93, into the primary tubing 92, and to the patient P. The combination of “negative pressure” and capillary forces will keep the saline S in the negative pressure chamber 20 from flowing into the conical extension 24.

The negative pressure chamber 20 allows all of the liquid medicine M to leave the secondary bag 70 and the drip chamber 12 and flow down the vertically intersecting tube 16, past the liquid level L, and to the terminal end 17. Once the last of the liquid medicine M reaches the terminal end 17, air from the drip chamber 12 will enter the bottom of the negative pressure chamber 20. Typically, this occurs through small air bubbles. Since the air from the drip chamber 12 is open to the ambient air, through the air vent 11 in the spike 10 and drip chamber 12, this will equalize the air pressure throughout the system and the air bubbles that come through the terminal end 17 will break the capillary bond at the terminal end 17 and between the outside wall 18 and inside wall 28 of the conical extension 24. Once this capillary bond is broken, the liquid saline solution S will begin to flow from the negative pressure chamber 20. Any air from the intersecting tube 16 will flow to the top of the negative pressure chamber 20 and will not enter the system. The flow of the liquid saline solution S will flush the liquid medicine M down the delivery tube 33. This will ensure that all of the residual liquid medicine M that is typically stranded in the delivery tube 33 is flushed from the system and is therefore fully used.

The present invention is well adapted to carry out the objectives and attain both the ends and the advantages mentioned, as well as other benefits inherent therein. While the present invention has been depicted, described, and is defined by reference to particular embodiments of the invention, such reference does not imply a limitation to the invention, and no such limitation is to be inferred. The depicted and described embodiments of the invention are exemplary only, and are not exhaustive of the scope of the invention. Consequently, the present invention is intended to be limited only be the spirit and scope of the claims, giving full cognizance to equivalents in all respects.

I claim:

1. A device to ensure the thorough delivery of liquid medicine in an intravenous delivery system having a primary saline drip bag with a primary delivery tube that delivers intravenous liquids to a patient and a secondary liquid medicine drip bag, said device consisting of:
a drip chamber having an integrated spike configured to attach to the secondary liquid medicine drip bag;
an intersecting tube functionally attached to said drip chamber;
a negative pressure chamber disposed below said drip chamber, said negative pressure chamber having a top with an opening disposed therein and an air opening attached thereto, said negative pressure chamber further having a bottom with a conical extension protruding downwardly therefrom and a secondary medicine delivery tube functionally attached from said conical extension to said primary delivery tube;
wherein said intersecting tube enters said negative pressure chamber through said opening, wherein said opening is sized to provide an air-tight seal around said intersecting tube, and wherein said intersecting tube terminates inside said conical extension;
wherein a saline solution is introduced into said negative pressure chamber and wherein said negative pressure chamber is sealed from ambient air to eliminate atmospheric pressure on the saline solution within said negative pressure chamber thereby preventing said saline solution from draining from said negative pressure chamber;
and wherein further when said spike is attached to said secondary liquid medicine drip bag said liquid medicine flows into said drip chamber, then into said intersecting tube and through said negative pressure chamber, then into said conical extension, and then into said secondary medicine delivery tube and on to said primary delivery tube and on to said patient;
and wherein when all of said liquid medicine has been drained from said secondary liquid medicine delivery bag and flowed through said intersecting tube ambient air is introduced into said negative pressure chamber from said intersecting tube thereby allowing said saline solution to drain from said negative pressure chamber and flush said liquid medicine from said secondary delivery tube, thereby ensuring thorough delivery of said liquid medicine to the patient.

2. The device to ensure the thorough delivery of liquid medicine of claim 1 wherein said negative pressure chamber is sealed from ambient air by a closable air opening.

3. The device to ensure the thorough delivery of liquid medicine of claim 2 wherein said saline solution is introduced into said negative pressure chamber by opening said closable air opening and lowering said negative pressure chamber below the primary saline drip bag to allow the saline from said primary saline drip bag to flow through said delivery tube into said secondary delivery tube and into said negative pressure chamber and then closing said closable air opening when the saline in said negative pressure chamber reaches a desired liquid level.

4. The device to ensure the thorough delivery of liquid medicine of claim 2 wherein said closable air opening consists of an attachable air-tight lid, said air-tight lid having a top opening configured to allow insertion of said intersecting tubing with an air-tight seal.

5. The device to ensure the thorough delivery of liquid medicine of claim 2 wherein said closable air opening consists of a needleless Luer Lock having a screw opening.

6. The device to ensure the thorough delivery of liquid medicine of claim 2 wherein said closable air opening consists of a snap-open cap.

7. The device to ensure the thorough delivery of liquid medicine of claim 1 wherein said intersecting tube has an outer wall having an outer diameter and wherein said conical extension has an inner wall having an inner diameter, wherein said inner diameter is greater than said outer diameter thereby creating a gap between said inner wall and said outer wall.

8. The device to ensure the thorough delivery of liquid medicine of claim 7 wherein said gap is between 1 mm and 6 mm.

9. The device to ensure the thorough delivery of liquid medicine of claim 7 wherein said gap is sized to ensure that capillary forces within said saline solution and between said outer wall and said inner wall are sufficient to hold said saline solution within said negative pressure chamber when said negative pressure chamber is sealed from ambient air.

10. A method for the complete delivery of intravenous (IV) medicine from a liquid medicine bag and secondary delivery tube to a patient, comprising the steps of:
providing a standard IV drip system with a primary IV bag containing a saline solution and a primary IV tube that runs from the primary IV bag to a patient, said primary IV tube including at least one standard Y-site attachment port;
catharizing a patient;
attaching said primary IV tube to said patient;
providing a secondary bag containing a liquid medicine;
attaching a drip chamber below said secondary bag;
connecting an intersecting tube below said drip chamber;
providing a negative pressure chamber having a top opening and a conical extension at the bottom, wherein said conical extension is attached to a secondary delivery tube;
inserting said intersecting tube through said top opening such that said intersecting tube terminates within said conical extension;
filling said negative pressure chamber with saline solution;
isolating said negative pressure chamber from atmospheric pressure to prevent said saline solution from draining from said negative pressure chamber;
releasing said liquid medicine from said secondary bag;
allowing said liquid medicine to drain into said drip chamber and through said intersecting tube, wherein said liquid medicine flows through said intersecting tube and into said conical extension, and wherein further said saline solution within said negative pressure chamber does not flow into said conical extension;
emptying said secondary bag of liquid medicine and thereby allowing ambient air to enter said negative pressure chamber thereby allowing said saline solution to drain from said negative pressure chamber, thereby flushing said liquid medicine from said secondary tube.

11. The method for complete delivery of an intravenous (IV) medicine from a liquid medicine bag and secondary delivery tube of claim 10, further comprising the steps of:
providing an openable and closable air opening to isolate said negative pressure chamber from atmospheric pressure.

12. The method for complete delivery of an intravenous (IV) medicine from a liquid medicine bag and secondary delivery tube of claim 11, wherein said openable and closeable air opening is an air-tight lid.

13. The method for complete delivery of an intravenous (IV) medicine from a liquid medicine bag and secondary delivery tube of claim 11, wherein said openable and closeable air opening is a Luer lock.

14. The method for complete delivery of an intravenous (IV) medicine from a liquid medicine bag and secondary delivery tube of claim 11, wherein said openable and closeable air opening is a snap-open cap.

15. The method for complete delivery of an intravenous (IV) medicine from a liquid medicine bag and secondary delivery tube of claim 10, further comprising the steps of:
defining an outer wall of said intersecting tube and an outer diameter of said outer wall;
defining an inner wall of said conical extension and an inner diameter of said inner wall;
wherein said inner diameter is greater than said outer diameter thereby creating a gap between said outer wall and said inner wall;
sizing said gap to ensure capillary forces in the saline solution within said negative pressure chamber are sufficient to overcome the forces of gravity when said negative pressure chamber is isolate from atmospheric pressure.
